(19) United States
(12) Patent Application Publication
(10) Pub. No.: US 2026/0078145 A1
(43) Pub. Date: Mar. 19, 2026

(54) Polypeptides having cholesterol-lowering function in soybean protein hydrolysate, preparation method thereof, and uses thereof

(71) Applicant: WUHAN ZHENFU INNOVATION BIOPHARMACEUTICAL CO., LTD, Wuhan (CN)

(72) Inventors: Yulin HE, Wuhan (CN); Yanshan DONG, Wuhan (CN); Chun WANG, Wuhan (CN); Ya GAO, Wuhan (CN); Yuanyuan LIAO, Wuhan (CN); Yupeng FAN, Wuhan (CN); Yefu WANG, Wuhan (CN)

(21) Appl. No.: 19/389,130

(22) Filed: Nov. 14, 2025

Related U.S. Application Data
(63) Continuation of application No. PCT/CN2024/073823, filed on Jan. 24, 2024.

(30) Foreign Application Priority Data
Jul. 12, 2023 (CN) 202310856234.5

(51) Int. Cl. C07K 7/06; A61K 38/00; A61P 3/06; C12N 9/50; C12N 9/54; C12P 21/06

(52) U.S. Cl. CPC C07K 7/06; A61P 3/06; C12N 9/50; C12N 9/54; C12P 21/06; C12Y 304/21062; A61K 38/00

(57) ABSTRACT

The present disclosure provides polypeptides having a cholesterol-lowering function in a soybean protein hydrolysate, a preparation method thereof, and uses thereof. The polypeptides are derived from peptide fragments having a molecular docking “binding energy” value of less than −1.2 kcal with cholesterol esterase. The peptide fragments of the polypeptides that are subjected to molecular docking with cholesterol esterase are derived from 23 polypeptides having a Peptide Ranker bioactivity score of greater than 0.9. The polypeptides possess a significant blood-lipid-lowering function.

### CROSS-REFERENCE TO RELATED APPLICATIONS

This application is a continuation of International Application No. PCT/CN2024/073823 filed on Jan. 24, 2024, which claims priority to Chinese Patent Application No. 202310856234.5, filed on Jul. 12, 2023. All of the aforementioned applications are incorporated herein by reference in their entireties.

### TECHNICAL FIELD

### Sequence Listing

This application contains a Sequence Listing which has been submitted in XML format via EFS-Web and is hereby incorporated by reference in its entirety. Said XML copy is named “POLYPEPTIDES HAVING CHOLESTEROL-LOWERING FUNCTION IN SOYBEAN PROTEIN HYDROLYSATE, PREPARATION METHOD THEREOF, AND USES THEREOF.xml”, created Nov. 12, 2025, and has a size of 21,176 bytes.

The present disclosure relates to the technical field of functional polypeptides, and specifically relates to polypeptides having a cholesterol-lowering function in a soybean protein hydrolysate, a preparation method thereof, and uses thereof.

### BACKGROUND

Dyslipidemia is associated with various human diseases. Currently, hyperlipidemia is becoming a disease that threatens national health and lowers the quality of life for the population. Research suggests that blood lipid-lowering mechanisms involve inhibiting the activity of pancreatic lipase to reduce the absorption of exogenous triglycerides, reducing the activity of fatty acid synthase to thereby decrease fat synthesis, and accelerating cholesterol metabolism to promote cholesterol excretion. Cholesterol esterase is an enzyme that catalyzes the hydrolysis of cholesterol esters into cholesterol and fatty acids. Cholesterol can be absorbed into the bloodstream through the formation of micelles and subsequent uptake by small intestinal epithelial cells. Excessive cholesterol in the blood can cause hyperlipidemia. Inhibiting the hydrolytic activity of cholesterol esterase can effectively reduce serum cholesterol, thereby lowering blood lipids.

Current drugs on the market are mainly statins and non-statin drugs. Statin drugs can stimulate an increase in blood sugar, leading to the risk of diabetes, whereas non-statin drugs are less effective than statins. Studies have found that protein components in natural foods can regulate dyslipidemia, and their blood lipid-lowering effects originate from their peptide fragments and amino acid residues. Developing safe and effective blood lipid-lowering active ingredients from natural food raw materials is of great significance for national health.

### Technical Problem

The technical problem to be solved by the present disclosure is how to provide a soybean protein hydrolysate having a good cholesterol-lowering effect.

### Technical Solution

In view of this, the present disclosure provides polypeptides having a cholesterol-lowering function in a soybean protein hydrolysate, a preparation method thereof, and uses thereof

In a first aspect, the present disclosure provides a polypeptide having a cholesterol-lowering function in a soybean protein hydrolysate, wherein the polypeptide is derived from a peptide fragment having a molecular docking “binding energy” value of less than −1.2 kcal with cholesterol esterase, and the peptide fragment of the polypeptide that is subjected to molecular docking with cholesterol esterase is derived from a polypeptide having a Peptide Ranker bioactivity score of greater than 0.9.

In a second aspect, the present disclosure provides a use of the polypeptide having a cholesterol-lowering function in a soybean protein hydrolysate according to the first aspect in preparation of a medicament for lowering hyperlipidemia, or a food or a health product for use in individuals with hyperlipidemia.

In a third aspect, the present disclosure provides a method for preparing a polypeptide having a cholesterol-lowering function in a soybean protein hydrolysate, wherein the method is used for preparing the polypeptide having a cholesterol-lowering function in a soybean protein hydrolysate according to the first aspect, comprising the following steps:
(1) preparing a protein hydrolysate: hydrolyzing a soybean protein with a protease to obtain the protein hydrolysate;
(2) determining cholesterol esterase inhibitory activity: performing a determination of cholesterol esterase inhibitory activity;
(3) identifying a sequence of the soybean protein hydrolysate: subjecting the soybean protein hydrolysate to liquid chromatography-tandem mass spectrometry (LC-MS/MS) determination to analyze the polypeptide composition of the soybean protein hydrolysate; and
(4) screening a cholesterol-lowering peptide: first screening identified polypeptide fragments to select potential bioactive peptides with Peptide Ranker score greater than 0.9, and then performing docking on the potential bioactive peptides using AutoDock 4.2.6 software to screen out the most potential cholesterol-lowering peptide.

### Beneficial Effects

The cholesterol-lowering peptides provided in the present disclosure are obtained by screening after hydrolyzing soybean protein with nattokinase. The obtained soybean protein hydrolysate possesses cholesterol esterase inhibitory activity. Sequencing of the hydrolysate identified 2359 polypeptides. All peptide fragments have a −10 log P value greater than 20, indicating high confidence. There are 23 peptide fragments meeting the Peptide Ranker score of greater than 0.9. After molecular docking of these 23 peptide fragments with cholesterol esterase, the molecular docking results show that the soybean peptide FFFPF has a “binding energy” value below −1.2 kcal, indicating that this peptide fragment has the best molecular docking effect with cholesterol esterase. Furthermore, animal experiments have verified that the peptide possesses a significant blood-lipid-lowering function. The cholesterol-lowering peptides provided in the present disclosure can be developed into hyperlipidemia-lowering drugs, and the active ingredients can be added into functional foods and health foods, showing good market prospects in the pharmaceutical and food industries.

### BRIEF DESCRIPTION OF THE DRAWINGS

To describe technical solutions in embodiments of the present disclosure more clearly, the following briefly introduces accompanying drawings required for describing the embodiments. Obviously, the drawings in the following description merely represent some embodiments of the present disclosure. Those skilled in the art may derive other drawings from these accompanying drawings without creative efforts.

FIG. 1 is a schematic diagram showing the interaction between the polypeptide FFFPF and cholesterol esterase molecules.

### DETAILED DESCRIPTION OF THE EMBODIMENTS

The following will provide a clear and complete description of the technical solutions in the embodiments of the present disclosure. Obviously, the described embodiments are only a part of the embodiments of the present disclosure, rather than all of them. All other embodiments obtained by those of ordinary skill in the art without creative efforts, based on the embodiments of the present disclosure, fall within the scope of protection of the present disclosure.

Unless otherwise specified, the experimental methods used in the following embodiments and examples are conventional methods. The nattokinase was prepared in the laboratory. All other materials and reagents used are reagents and materials obtainable through commercial sources. Unless otherwise specified, all equipment used is conventional experimental equipment.

### Embodiment 1 Preparation of Soybean Protein Hydrolysate

Soybean protein was used as a raw material and dissolved in pure water to prepare a 5% (weight/volume, W/V) soybean protein isolate solution. The solution was stirred at 90° C. for 15 minutes. After cooling to 45° C., nattokinase was added at an enzyme-to-substrate ratio of 6000 IU/g. After hydrolyzing with stirring for 2 hours, the temperature was raised to 90° C. for 15 minutes to inactivate the enzyme. After stopping the hydrolysis, the mixture was centrifuged at 8000 revolutions per minute (rpm) for 20 minutes. The resulting supernatant was the soybean protein hydrolysate.

### Embodiment 2 Preparation of Soybean Protein Hydrolysate Using Alkaline Protease

The procedure was the same as in Embodiment 1, except that nattokinase was replaced with alkaline protease.

### Embodiment 3 Determination of Cholesterol Esterase Inhibitory Activity of Soybean Protein Hydrolysates

In a 96-well microplate, 50 μL of the hydrolysate from Embodiment 1, 50 μL of the hydrolysate from Embodiment 2, 50 μL of a 25 μg/mL cholesterol esterase solution, and 50 μL of a 10 mM p-nitrophenyl butyrate solution as substrate were incubated at 25° C. for 5 minutes in a phosphate buffer (pH 7.0) containing 100 mM NaCl and 5.16 mM sodium taurocholate. The absorbance was recorded at 405 nm using a microplate reader. Simvastatin was used as a positive control. The cholesterol esterase inhibitory activity was calculated according to Formula (1):

$$Cholesterol Esterase Activity Inhibitory Rate / \% = ( 1 - \frac{C - D}{A - B} ) \times 100 \% \qquad Formula ( 1 )$$

Where, in Formula (1):
A represents the blank absorbance; B represents the blank control absorbance; C represents the sample absorbance; D represents the sample blank absorbance.

The test results are shown in Table 1. The hydrolysate from Embodiment 1 (soybean protein hydrolyzed with nattokinase) exhibited higher inhibitory activity against cholesterol esterase than the hydrolysate from Embodiment 2 (soybean protein hydrolyzed with alkaline protease). This indicates that the hydrolysate obtained in Embodiment 1 has potentially superior cholesterol-lowering efficacy compared to Embodiment 2. This difference may be related to the peptide fragments in the hydrolyzed protein solutions, wherein Embodiment 1 is more likely to contain cholesterol-lowering peptides.

TABLE 1
Inhibitory Activity of Hydrolysates on Cholesterol Esterase

| Sample | Inhibition Rate (%) |
|---|---|
| Embodiment 1 | 38.23 ± 1.02c |
| Embodiment 2 | 20.47 ± 1.11b |
| Lovastatin | 82.06 ± 0.61a |

### Embodiment 4 LC-MS/MS Analysis of Polypeptide Sequences

The soybean protein hydrolysate was transferred to a 10 kDa molecular weight cut-off (MWCO) ultrafiltration centrifuge tube and centrifuged at 12000×g for 10 minutes. The ultrafiltered solution was desalted using a C18 desalting column. The sample was eluted with Elution Buffer (0.1% Formic Acid (FA), 60% Acetonitrile (ACN)), and the eluate was transferred to a new Eppendorf (EP) tube. The eluted sample was centrifuged, concentrated, and dried for subsequent mass spectrometry analysis. After desalting, the centrifuged and dried sample was redissolved in 100 μL of Nano-LC Mobile Phase A (0.1% FA/water), loaded into vials and injected for online LC-MS analysis. The dissolved sample was loaded onto a nanoViper C18 pre-column (3 μm, 100 Å) with a volume of 2 μL, followed by washing and desalting with a volume of 20 μL. Liquid chromatography was performed using an Easy nLC 1200 nanoflow liquid chromatography system (ThermoFisher, USA). After the sample was desalted and retained on the pre-column, it was separated on an analytical column. The specifications of the analytical column were a C18 reversed-phase chromatographic column (Acclaim PepMap RSLC, 75 μm×25 cm, C18, 2 μm, 100 Å). The gradient used in the experiment involved an increase of Mobile Phase B (80% ACN, 0.1% FA) from 5% to 38% within 30 minutes. Mass spectrometry was performed using a ThermoFisher Q Exactive system (ThermoFisher, USA) coupled with a NanoFlex ion source (ThermoFisher, USA) for nanospray. The spray voltage was set to 1.9 kV, and the ion transfer tube temperature was set to 275° C. The mass spectrometry scanning was conducted in Data Dependent Acquisition (DDA) mode. The full scan (MS1) resolution was set to 70000, with a scan range of 100-1500 m/z and a maximum injection time of 100 ms. A maximum of 20 precursor ions with charge states of 1+ to 3+ were selected for fragmentation per DDA cycle. The maximum injection time for MS/MS (MS2) scans was 50 ms. The normalized collision energy (using Higher-energy C-trap Dissociation, HCD) was set to 28 eV for all precursor ions. The dynamic exclusion was set to 6 seconds. The raw data files acquired by mass spectrometry were processed and analyzed using PEAKS Studio 8.5 software (Bioinformatics Solutions Inc., Waterloo, Canada). The database used for search was the target species protein database downloaded from UniProt. The search parameters were set as follows: precursor mass tolerance was 10 ppm, and fragment mass tolerance was 0.05 Da. Variable modifications included Oxidation (M), Acetylation (Protein N-term), Deamidation (NQ), Pyro-glu from E, and Pyro-glu from Q.

### Embodiment 5 Screening of Cholesterol-Lowering Peptides Based on Molecular Docking Technology

First, the peptide fragments identified by LC-MS/MS described above were preliminarily screened using Peptide Ranker to select peptide fragments having a bioactivity prediction score of greater than 0.9. These selected peptide fragments were then subjected to molecular docking with cholesterol esterase. A lower “binding energy” value indicates better docking results and suggests greater cholesterol-lowering potential. The peptide fragments with a bioactivity prediction score greater than 0.9 are shown in Table 2.

TABLE 2

| | Bioactivity |
|---|---|
| | Prediction |
| Polypeptide | Score |
| FFFPF | 0.998297 |
| DFPALWLL | 0.961289 |
| GNWGPL | 0.951482 |
| NWGPL | 0.948794 |
| NNPFKF | 0.937992 |
| LFLPHF | 0.933212 |
| GQYPYGGF | 0.922718 |
| NNNPFKFL | 0.916986 |
| NNPFSF | 0.915526 |
| WNPNNKPF | 0.913682 |
| NNNPFKF | 0.910358 |
| GSGIFGM | 0.909999 |
| QQDEDEDEDEDDEDE | 0.909957 |
| QIPSHPPRRPSHGK | |
| DEDEDEDEDDEDEQI | 0.907408 |
| PSHPPRRPSHGK | |
| QDEDEDEDEDDEDEQ | 0.907025 |
| IPSHPPRRPSHGK | |
| LRPPF | 0.955234 |
| FSEYPPLGRF | 0.936563 |
| FEFLPPFIRR | 0.934714 |
| NEPWWPKL | 0.925473 |
| VFDPF | 0.923523 |
| SLDVWDPFEGFPF | 0.915986 |
| HFGSF | 0.915596 |
| FFYNPK | 0.902779 |

The crystal structure of cholesterol esterase (PDB ID: 1F6W) was obtained from the research collaboratory for structural bioinformatics (RCSB) database (http://www.resb.org/). Semi-flexible molecular docking between the peptides with bioactivity prediction scores higher than 0.90 and cholesterol esterase was performed using AutoDock 4.2.6 software. The docking scores are shown in Table 2.

The molecular docking scores for the potential cholesterol-lowering peptides with cholesterol esterase are shown in Table 3.

### Table 3

| | Binding |
|---|---|
| | energy/ |
| Polypeptide | kcal |
| FFFPF | −2.05 |
| DFPALWLL | 1.21 |
| GNWGPL | 3.72 |
| NWGPL | 3.05 |
| NNPFKF | 0.69 |
| LFLPHF | 0.03 |
| GQYPYGGF | 2.19 |
| NNNPFKFL | 4.19 |
| NNPFSF | −0.12 |
| WNPNNKPF | 2.43 |
| NNNPFKF | 4.08 |
| GSGIFGM | 2.19 |
| QQDEDEDEDEDDEDE | 3.02 |
| QIPSHPPRRPSHGK | |
| DEDEDEDEDDEDEQI | 3.18 |
| PSHPPRRPSHGK | |
| QDEDEDEDEDDEDEQ | 3.26 |
| IPSHPPRRPSHGK | |
| LRPPF | −0.28 |
| FSEYPPLGRF | 7.74 |
| FEFLPPFIRR | 7.88 |
| NEPWWPKL | 7.1 |
| VFDPF | 1.14 |
| SLDVWDPFEGFPF | 7.29 |
| HFGSF | 0.5 |
| FFYNPK | 0.9 |

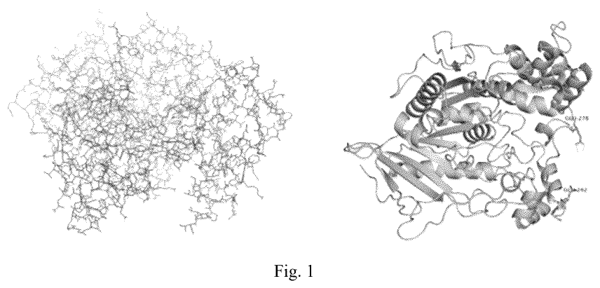

As shown in FIG. 1, which is a schematic diagram of the interaction between the polypeptide FFFPF and cholesterol esterase molecules, the polypeptide FFFPF underwent semi-flexible docking with cholesterol esterase. Other key amino acid residues of cholesterol esterase interacting with the polypeptide included GLU242 and GLU278. Interaction forces included hydrogen bonds and Pi-C bonds, among others. The polypeptide FFFPF is considered a potential cholesterol-lowering peptide.

### Embodiment 6 Animal Experiment on Lipid-Lowering Effects of Cholesterol-Lowering Peptide

The cholesterol-lowering peptide FFFPF was prepared using Fmoc solid-phase synthesis. Analysis by High-Performance Liquid Chromatography (HPLC) and mass spectrometry confirmed a peptide purity greater than 9500.

A hyperlipidemic animal model was established as follows: Forty healthy adult male Sprague-Dawley (SD) rats, weighing 200±20 g, were used. A blank control group of 10 rats was fed a standard diet. The remaining 30 rats constituted the modeling group and were fed a high-fat diet (commercially available, 60% fat content).

After 1-2 weeks of feeding the high-fat diet to the modeling group, blood was collected from the blank control group and the modeling group without fasting (from the inner canthus or tail). Serum was separated as soon as possible after blood collection, and serum levels of Total Cholesterol (TC), Triglycerides (TG), Low-Density Lipoprotein Cholesterol (LDL-C), and High-Density Lipoprotein Cholesterol (HDL-C) were measured. The modeling group was randomly divided into 3 groups (model control group, cholesterol-lowering peptide group, lovastatin control group). No significant differences in TC, TG, LDL-C, and HDL-C were observed among these groups. Compared with the blank control group, each group of the modeling group showed significantly increased TC, TG, and LDL-C, indicating successful model establishment.

The blank control group and the model control group were administered an equal volume of 0.9% physiological saline by oral gavage. The other two groups were administered aqueous solutions of the test samples by oral gavage. The gavage doses are listed in Table 4.

TABLE 4
Dosage for Each Group by Oral Gavage

| Group | Dosage/mg · kg⁻¹ |
|---|---|
| Blank Control Group | — |
| Model Control Group | — |
| Cholesterol-Lowering | 800 mg/kg |
| Peptide | |
| Lovastatin Control Group | 0.21 mg/kg |

During the gavage period, the blank control group was fed a standard diet daily, while the other groups continued receiving the high-fat diet. All rats had free access to water. Oral gavage was performed once daily for 4 consecutive weeks, and body weights were measured regularly. At the end of the 4 weeks, blood was collected from the orbital sinus without fasting, and serum was separated promptly. Serum levels of TC, TG, LDL-C, and HDL-C were measured.

TABLE 5
Changes in Rat Body Weight

| | Body Weight (g) | | |
|---|---|---|---|
| | At Day 0 of | At Day 14 of | At Day 28 of |
| Group | Administration | Administration | Administration |
| Blank Control Group | 201.23 | 220.46 | 237.13 |
| Model Control Group | 202.28 | 219.24 | 236.60 |
| Cholesterol-Lowering | 200.89 | 220.75 | 237.35 |
| Peptide | | | |
| Lovastatin Control | 200.78 | 221.16 | 236.39 |
| Group | | | |

The body weight results in Table 5 show no significant differences in body weight among the groups at Day 14 and Day 28 of administration, indicating that the cholesterol-lowering peptide, lovastatin, and the treatments administered to the blank control group and the model control group had no significant effect on rat body weight.

TABLE 6
Serum Index Detection Results in Rats After 4 Weeks of Oral Gavage

| | | | Low-Density | |
|---|---|---|---|---|
| | | Total | Lipoprotein | High-Density |
| | Triglycerides | Cholesterol | Cholesterol | Lipoprotein |
| Group | (TG) | (TC) | (LDL-C) | Cholesterol (HDL-C) |
| Blank Control | 0.85 | 2.16 | 0.21 | 0.58 |
| Group | | | | |
| Model Control | 1.51 | 5.09 | 0.89 | 0.32 |
| Group | | | | |
| Cholesterol-Low | 1.01** | 3.19** | 0.38** | 0.52** |
| ering Peptide | | | | |
| Lovastatin | 0.98** | 3.06** | 0.32** | 0.54** |
| Control Group | | | | |

Note:
**P < 0.01 compared with the Model Control Group.

As can be seen from Table 6, compared with the model control group, the cholesterol-lowering peptide group, after 4 weeks of oral gavage, significantly reduced serum TG, TC, and LDL-C, showing highly significant differences (**P<0.01). The cholesterol-lowering peptide group also increased serum HDL-C levels, showing a highly significant difference compared to the model control group (**P<0.01). No significant differences were observed in the serum indices between the cholesterol-lowering peptide group and the lovastatin control group. However, both treatment groups showed significant changes compared to the model control group. These results indicate that the cholesterol-lowering peptide screened in the present disclosure can effectively reduce serum TG, TC, and LDL-C levels in hyperlipidemic rats, with an efficacy comparable to lovastatin, demonstrating a good blood-lipid-lowering effect.

The foregoing provides a detailed description of polypeptides having a cholesterol-lowering function in a soybean protein hydrolysate, a preparation method thereof, and uses thereof, as provided by the embodiments of the present disclosure. Specific examples are used herein to illustrate the principles and embodiments of the present disclosure. The description of the foregoing embodiments is intended only to aid in understanding the method and core concepts of the present disclosure. Furthermore, for those skilled in the art, based on the ideas of the present disclosure, changes may be made to the specific embodiments and scope of application. In summary, the content of this specification should not be construed as limiting the present disclosure.

What is claimed is:

1. A polypeptide having a cholesterol-lowering function in a soybean protein hydrolysate, wherein the polypeptide is derived from a peptide fragment having a molecular docking “binding energy” value of less than −1.2 kcal with cholesterol esterase, and the peptide fragment of the polypeptide that is subjected to molecular docking with cholesterol esterase is derived from a polypeptide having a Peptide Ranker bioactivity score of greater than 0.9.

2. The polypeptide having a cholesterol-lowering function in a soybean protein hydrolysate according to claim 1, wherein the amino acid sequence of the polypeptide is at least one selected from the group consisting of FFFPF, DFPALWLL, GNWGPL, NWGPL, NNPFKF, LFLPHF, GQYPYGGF, NNNPFKFL, NNPFSF, WNPNNKPF, NNNPFKF, GSGIFGM, QQDEDEDEDEDDEDEQIPSHPPRRPSHGK, DEDEDEDEDDEDEQIPSHPPRRPSHGK, QDEDEDEDEDDEDEQIPSHPPRRPSHGK, LRPPF, FSEYPPLGRF, FEFLPPFIRR, NEPWWPKL, VFDPF, SLDVWDPFEGFPF, HFGSF, and FFYNPK.

3. The polypeptide having a cholesterol-lowering function in a soybean protein hydrolysate according to claim 1, wherein the amino acid sequence of the polypeptide is FFFPF.

4. Use of the polypeptide having a cholesterol-lowering function in a soybean protein hydrolysate according to claim 1 in preparation of a medicament for lowering hyperlipidemia, or a food or a health product for use in individuals with hyperlipidemia.

5. A method for preparing a polypeptide having a cholesterol-lowering function in a soybean protein hydrolysate, wherein the method is used for preparing the polypeptide having a cholesterol-lowering function in a soybean protein hydrolysate according to claim 1, comprising the following steps:
(1) preparing a protein hydrolysate: hydrolyzing a soybean protein with a protease to obtain the protein hydrolysate;
(2) determining cholesterol esterase inhibitory activity: performing a determination of cholesterol esterase inhibitory activity;
(3) identifying a sequence of the soybean protein hydrolysate: subjecting the soybean protein hydrolysate to liquid chromatography-tandem mass spectrometry (LC-MS/MS) determination to analyze the polypeptide composition of the soybean protein hydrolysate; and
(4) screening a cholesterol-lowering peptide: first screening identified polypeptide fragments to select potential bioactive peptides with Peptide Ranker score greater than 0.9, and then performing docking on the potential bioactive peptides using AutoDock 4.2.6 software to screen out the most potential cholesterol-lowering peptide.

6. The method for preparing a polypeptide having a cholesterol-lowering function in a soybean protein hydrolysate according to claim 5, wherein the protease is nattokinase, with an enzyme-to-substrate ratio of 6000 IU/g.

7. The method for preparing a polypeptide having a cholesterol-lowering function in a soybean protein hydrolysate according to claim 5, wherein the cholesterol esterase is derived from the research collaboratory for structural bioinformatics (RCSB) database (PDB: 1F6W).

8. The method for preparing a polypeptide having a cholesterol-lowering function in a soybean protein hydrolysate according to claim 5, comprising the following steps:
(1) dissolving the soybean protein: using the soybean protein as a raw material, dissolving the soybean protein in pure water to prepare a 3-7% (W/V) soybean protein isolate solution;
(2) enzymatically hydrolyzing the soybean protein: adding the enzyme to the solution, and performing enzymatic hydrolysis with stirring at a certain temperature to obtain a hydrolyzed protein solution;
(3) obtaining the soybean protein hydrolysate: inactivating the enzyme, performing centrifugation, and taking a supernatant to obtain the soybean protein isolate hydrolysate;
(4) determining cholesterol esterase inhibitory activity: performing the determination of cholesterol esterase inhibitory activity with reference to a literature method;
(5) identifying a polypeptide sequence: performing peptide mapping identification on the hydrolysate of the enzymatically hydrolyzed soybean protein using a ThermoFisher Q Exactive system; and
(6) screening the cholesterol-lowering peptide: first screening identified polypeptide fragments to select potential bioactive peptides with Peptide Ranker score greater than 0.9, and then performing molecular docking on the potential bioactive peptides using AutoDock 4.2.6 software to screen out the most potential cholesterol-lowering peptide.

9. The method for preparing a polypeptide having a cholesterol-lowering function in a soybean protein hydrolysate according to claim 8, wherein in step (1), the soybean protein isolate is dissolved by stirring at 90° C. for 15 minutes; and in step (2), the temperature is 45° C., and the stirring time is 2 hours.

10. The method for preparing a polypeptide having a cholesterol-lowering function in a soybean protein hydrolysate according to claim 8, wherein in step (3), the enzyme inactivation temperature is 90° C., and the enzyme inactivation time is 15 minutes; and in step (4), the centrifugation speed is 5000-10000 rpm, and the centrifugation time is 20 minutes.


SEQUENCE LISTING (23 sequences)

SEQ ID NO: 1 - protein, 5 aa, synthetic construct
FFFPF

SEQ ID NO: 2 - protein, 8 aa, synthetic construct
DFPALWLL

SEQ ID NO: 3 - protein, 6 aa, synthetic construct
GNWGPL

SEQ ID NO: 4 - protein, 5 aa, synthetic construct
NWGPL

SEQ ID NO: 5 - protein, 6 aa, synthetic construct
NNPFKF

SEQ ID NO: 6 - protein, 6 aa, synthetic construct
LFLPHF

SEQ ID NO: 7 - protein, 8 aa, synthetic construct
GQYPYGGF

SEQ ID NO: 8 - protein, 8 aa, synthetic construct
NNNPFKFL

SEQ ID NO: 9 - protein, 6 aa, synthetic construct
NNPFSF

SEQ ID NO: 10 - protein, 8 aa, synthetic construct
WNPNNKPF

SEQ ID NO: 11 - protein, 7 aa, synthetic construct
NNNPFKF

SEQ ID NO: 12 - protein, 7 aa, synthetic construct
GSGIFGM

SEQ ID NO: 13 - protein, 29 aa, synthetic construct
QQDEDEDEDEDDEDEQIPSHPPRRPSHGK

SEQ ID NO: 14 - protein, 27 aa, synthetic construct
DEDEDEDEDDEDEQIPSHPPRRPSHGK

SEQ ID NO: 15 - protein, 28 aa, synthetic construct
QDEDEDEDEDDEDEQIPSHPPRRPSHGK

SEQ ID NO: 16 - protein, 5 aa, synthetic construct
LRPPF

SEQ ID NO: 17 - protein, 10 aa, synthetic construct
FSEYPPLGRF

SEQ ID NO: 18 - protein, 10 aa, synthetic construct
FEFLPPFIRR

SEQ ID NO: 19 - protein, 8 aa, synthetic construct
NEPWWPKL

SEQ ID NO: 20 - protein, 5 aa, synthetic construct
VFDPF

SEQ ID NO: 21 - protein, 13 aa, synthetic construct
SLDVWDPFEGFPF

SEQ ID NO: 22 - protein, 5 aa, synthetic construct
HFGSF

SEQ ID NO: 23 - protein, 6 aa, synthetic construct
FFYNPK